(19) United States
(12) Patent Application Publication
(10) Pub. No.: US 2024/0058448 A1
(43) Pub. Date: Feb. 22, 2024

(54) Long Acyl-Chain Phospholipid Oxygen Microbubbles for Treating Tumor Hypoxia

(71) Applicant: THE REGENTS OF THE UNIVERSITY OF COLORADO, A BODY CORPORATE, Denver, CO (US)

(72) Inventors: Mark A. Borden, Boulder, CO (US); Traci D. Reusser, Superior, CO (US)

(21) Appl. No.: 17/766,376

(22) PCT Filed: Sep. 30, 2020
(86) PCT No.: PCT/US2020/053622

§ 371 Date: Apr. 4, 2022

Related U.S. Application Data
(60) Provisional application No. 62/910,991, filed on Oct. 4, 2019.

(51) Int. Cl. A61K 41/00; A61K 9/107; A61K 47/10; A61K 47/24; A61N 7/00; A61N 5/10

(52) U.S. Cl. CPC A61K 41/0033; A61K 9/1075; A61K 47/10; A61K 47/24; A61N 7/00; A61N 5/10; A61N 2007/0039; A61N 2005/1098

(57) ABSTRACT

The present invention includes system, methods, and compositions for the generation of novel oxygen microbubbles, and in particular oxygen microbubbles with a longer acyl-chain phospholipid for increased circulation persistence and oxygen payload. The novel oxygen microbubbles of the invention may be particularly suited for the treatment of tumor hypoxia in radiation oncology.

This International PCT Application claims the benefit of and priority to U.S. Provisional Application No. 62/910,991, filed Oct. 4, 2019. The entire specification and figures of the above-referenced application is hereby incorporated, in its entirety by reference.

### GOVERNMENT INTEREST

This invention was made with government support under grant number CA232148 awarded by the National Institutes of Health. The government has certain rights in the invention.

### TECHNICAL FIELD

The present invention includes system, methods, and compositions for the generation of novel oxygen microbubbles, and in particular oxygen microbubbles with a longer acyl-chain phospholipid for increased circulation persistence and oxygen payload. The novel oxygen microbubbles of the invention may be particularly suited for the treatment of tumor hypoxia in radiation oncology.

### BACKGROUND

In recent work, oxygen microbubbles (OMB) have been used for oxygenation of hypoxic tumors for radiotherapy treatment. Relieving hypoxia in the tumor with ultrasound-guided OMB destruction during radiotherapy could significantly improve the treatment outcome. Additionally, ultrasound can be used for real-time visualization of the presence of OMB at the tumor location. Ultrasound-induced destruction of OMB has been shown to increase dissolved oxygen content in vitro and increase in vivo oxygen levels and tumor control in a fibrosarcoma rodent model when injected directly into the tumor. However, an intravenous injection, rather than intra-tumoral injection, of OMB would be highly advantageous for clinical translation. Unfortunately, conventional OMB have short circulation lifetimes and may be ineffective.

However, the design of the microbubble shell composition and gas core, can improve the circulation of the microbubble. To overcome these design issues, research has focused on increasing lipid acyl-chain length to create a more stable OMB for increased circulation persistence and oxygen payload. OMB have a phospholipid outer shell with a polyethylene glycol (PEG) brush that contains an oxygen gas core. Size-selection of the oxygen microbubbles has been resolved to achieve microbubbles that are approximately 2-10 μm in diameter and have been observed with ultrasound after being intravenously injected.

Despite these advances in OMB design, there still exists a need for a novel and effective OMB having increased stability, circulation persistence, and oxygen payload that addresses the concerns outlined above.

### SUMMARY OF THE INVENTION(S)

One aspect of the inventive technology includes the production of novel OMB that exhibit an increase in contrast persistence and circulation in vivo. In one preferred aspect, OMB were designed to include a plurality of long acyl-chain phospholipids configured to improve microbubble stability for oxygen delivery to tissues and cells, and preferably to hypoxic tumors.

Another aspect of the current inventive technology may include an OMB having a lipid shell coupled with a long acyl chain. In a preferred embodiment, the lipid may include a phospholipid, or other hydrophobic molecules, and the acyl chain may be less than 24 carbons long.

Another aspect of the current inventive technology may include the diagnostic use of OMB having a long acyl-chain phospholipid shell, wherein the long acyl-chain phospholipid shell is configured to increase microbubble's stability, particularly in in vivo environments, and allow for the microbubble to have an increased surface to volume ratio resulting in larger microbubbles. Another aspect of the current inventive technology may include the methods of production and isolation of OMB having a long acyl-chain phospholipid shell, where such OMB are preferably between 2-10 μm in diameter.

Another aspect of the current inventive technology may include the diagnostic use of OMB having a long acyl-chain phospholipid shell. In one preferred embodiment, an OMB produced with 1,2-dibehenoyl-sn-glycero-3-phosphocholine (C22:0) shells may be visible by ultrasound and enhance ultrasound efficiency by creating a measurable increase in contrast intensity.

Another aspect of the current inventive technology may include the therapeutic use of OMB having a long acyl-chain phospholipid shell for the treatment of hypoxic tumors. In a preferred embodiment, the delivery of oxygen by the invention's OMB may increase the effectiveness of radiation, and in particular x-ray radiation treatment of hypoxic tumors.

Another aspect of the current inventive technology may include the therapeutic use of OMB having a long acyl-chain phospholipid shell configured for intravenous administration rather than, for example intra-tumoral, injection. In this embodiment, oxygenation of hypoxic tumors may be accomplished through the administration of a therapeutically effective amount of OMB having a long acyl-chain phospholipid shell.

Another aspect of the current inventive technology includes kits and methods of using OMB having a long acyl-chain phospholipid shell that may be used in ultrasound-based diagnostic and therapeutic technologies, such as ultrasound imaging, drug delivery and ultrasound-induced drug delivery.

Another aspect of the current inventive technology includes kits and methods of using OMB having a long acyl-chain phospholipid shell that may be used in radiation-based cancer treatments. Another aspect of the invention may include the co-administration of OMB having a long acyl-chain phospholipid shell and a secondary cancer therapeutic treatment, such as chemotherapeutic or radiation treatments.

Another aspect of the current inventive technology includes kits and methods of using OMB having a long acyl-chain phospholipid shell that may be used in anti-bacterial treatments, and preferably therapeutic treatments directed to treating bacterial biofilms. Another aspect of the invention may include the co-administration of OMB having a long acyl-chain phospholipid shell and an antibacterial treatment, such as an antibiotic.

Another aspect of the current inventive technology includes kits and methods of using OMB having a long acyl-chain phospholipid shell that may be used in intravenous delivery systems.

Another aspect of the current inventive technology includes methods of treating an individual comprising administering a therapeutically effective amount of OMB having a long acyl-chain phospholipid shell to an individual in need of thereof, the acyl-chain having less than 24 carbons, and preferably 22 carbons.

Additional aspects of the invention may include one or more of the following preferred embodiments:
1. A novel microbubble composition comprising a lipid outer shell encasing an gas core wherein said lipid outer shell comprises a plurality of long acyl-chain phospholipids configured to improve microbubble stability.
2. The composition of embodiment 1 and further comprising a polyethylene glycol (PEG) brush structure.
3. The composition of embodiment 1 wherein said plurality of long acyl-chain phospholipids configured to improve microbubble stability comprises a plurality of long acyl-chain phospholipids having less than 24 carbons in the acyl-chain.
4. The composition of embodiment 1 wherein said plurality of long acyl-chain phospholipids configured to improve microbubble stability comprises a plurality of long acyl-chain phospholipids having at least 22 carbons in the acyl-chain.
5. The composition of embodiment 1 wherein said long acyl-chain phospholipid comprises 1,2-dibehenoyl-sn-glycero-3-phosphocholine (DBPC).
6. The composition of embodiment 1 wherein said long acyl-chain phospholipid comprises 1,2-distearoyl-sn-gylcero-3-phosphocholine (DSPC)
7. The composition of embodiment 1 and 5 wherein said microbubble is formed using an emulsifier.
8. The composition of embodiment 7 wherein said emulsifier comprises 1,2-distearoyl-sn-glycero-3-phosphoethanolamine-N-(polyethylene glycol 2000) (DSPE-PEG2000).
9. The composition of embodiments 1 and 5 wherein said novel microbubble comprises a microbubble having increased ultrasound contrast enhancement and persistence in vivo and in vitro.
10. The composition of embodiment 1, 5 and 9 wherein said gas core comprises an oxygen gas core.
11. The composition of embodiment 10 wherein said oxygen gas core comprises a pure oxygen gas core.
12. The composition of embodiment 1, 5 and 11 wherein said novel microbubble is between 0.1-10 μm in diameter.
13. The composition of embodiment 1, 5 and 11 wherein said novel microbubble is larger than 10 μm in diameter.
14. Administering a therapeutically effective amount of at least one of the compositions of embodiments 1-13 to a patient in need thereof.
15. The method of embodiment 14 wherein said step of administering comprises administering a therapeutically effective amount of at least one of the compositions of embodiments 1-13 to a subject in need thereof to treat a hypoxic tumor.
16. The method of embodiment 14 wherein said step of administering comprises administering a therapeutically effective amount of at least one of the compositions of embodiments 1-13 to a subject in need thereof to treat a bacterial infection.
17. The method of embodiment 16 wherein said bacterial infection comprises a bacterial biofilm.
18. The method of embodiment 14 wherein said step of administering comprises administering a therapeutically effective amount of at least one of the compositions of embodiments 1-13 intravenously to a subject in need thereof.
19. The method of embodiment 14 wherein said step of administering comprises administering a therapeutically effective amount of at least one of the compositions of embodiments 1-13 intravenously to a subject in need thereof, and further applying ultrasound radiation to said composition in vivo.
20. The method of embodiment 14 wherein said step of administering comprises administering a therapeutically effective amount of at least one of the compositions of embodiments 1-13 intravenously to a subject in need thereof, wherein said subject has a hypoxic tumor that is oxygenated by at least one of said compositions, and further applying therapeutic radiation to said oxygenated hypoxic tumor.
21. The method of embodiment 21 wherein said therapeutic radiation comprises x-ray radiation.

Additional aspects of the invention may be evidenced from the specification, claims and figures provided below.

### BRIEF DESCRIPTION OF THE DRAWINGS

The above and other aspects, features, and advantages of the present disclosure will be better understood from the following detailed descriptions taken in conjunction with the accompanying figures, all of which are given by way of illustration only, and are not limiting the presently disclosed embodiments, in which:

FIG. 1. Cartoon illustration of OMB designed for in vivo applications.

FIG. 2. Multisizer III number weighted size distributions for size-selected: A and C. DSPC (C18:0) and B and D. DBPC (C22:0) OMB.

FIG. 3. Contrast enhancement in the kidney: A. before and B. after OMB administration.

FIG. 4. Time intensity curve for DBPC OMB volume fraction study.

### DETAILED DESCRIPTION OF THE INVENTION(S)

The present invention provides for novel microbubble compositions, and preferably oxygen microbubbles (OMB). The term microbubbles refers to vesicles which are generally characterized by the presence of one or more membranes or walls or shells surrounding an internal void that is filled with a gas or precursor thereto. In some embodiments, the microbubbles comprise one or more lipids. The term lipids includes agents exhibiting amphipathic characteristics causing it to spontaneously adopt an organized structure in water wherein the hydrophobic portion of the molecule is sequestered away from the aqueous phase.

In one aspect the invention provides gas filled microbubbles. In some embodiments the microbubbles comprise one or more gases inside a lipid shell. In some embodiments, the lipid shell comprises one or more polymerizable lipids, such as a phospholipid. In some embodiments, the invention provides gas filled microbubbles substantially devoid of liquid in the interior. In some embodiments, the microbubbles are at least about 90% devoid of liquid, at least about 95% devoid of liquid, or about 100% devoid of liquid.

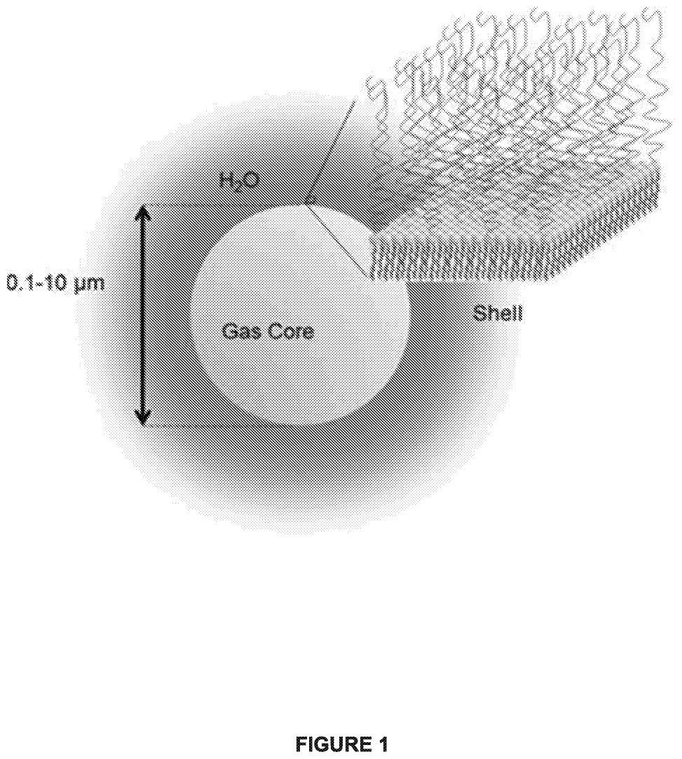

The microbubbles of the current invention may, in a preferred embodiment include an oxygen gas core as shown on FIG. 1. In alternative embodiments, microbubbles having long acyl chain phospholipids included in this description may contain any combination of gases suitable for the diagnostic or therapeutic method desired. For example, various biocompatible gases such as air, nitrogen, carbon dioxide, argon, xenon, neon, helium, and/or combinations thereof may be employed. Other suitable gases will be apparent to those skilled in the art, the gas chosen being only limited by the proposed application of the microbubbles. In some embodiments, the microbubbles contain gases with high molecular weight and size. In some embodiments, the microbubbles contain fluorinated gases, fluorocarbon gases, and perfluorocarbon gases. In some embodiments, the perfluorocarbon gases include perfluoropropane, perfluorobutane, perfluorocyclobutane, perfluoromethane, perfluoroethane and perfluoropentane, especially perfluoropropane. In some embodiments, the perfluorocarbon gases have less than six carbon atoms. Gases that may be incorporated into the microbubbles include but are not limited to: SF6, CF4, C2F6, C3F6, C3F8 C4F6, C4F8, C4F10, C5F10, C5F12, C6F12, (1-trifluoromethyl), propane (2-trifluoromethyl)-1,1,1,3,3,3 hexafluoro, and butane (2-trifluoromethyl)-1,1,1,3,3,3,4,4,4 nonafluor, air, oxygen, nitrogen, carbon dioxide, noble gases, vaporized therapeutic compounds, and mixtures thereof. The halogenated versions of hydrocarbons, where other halogens are used to replace F (e.g., Cl, Br, I) would also be useful.

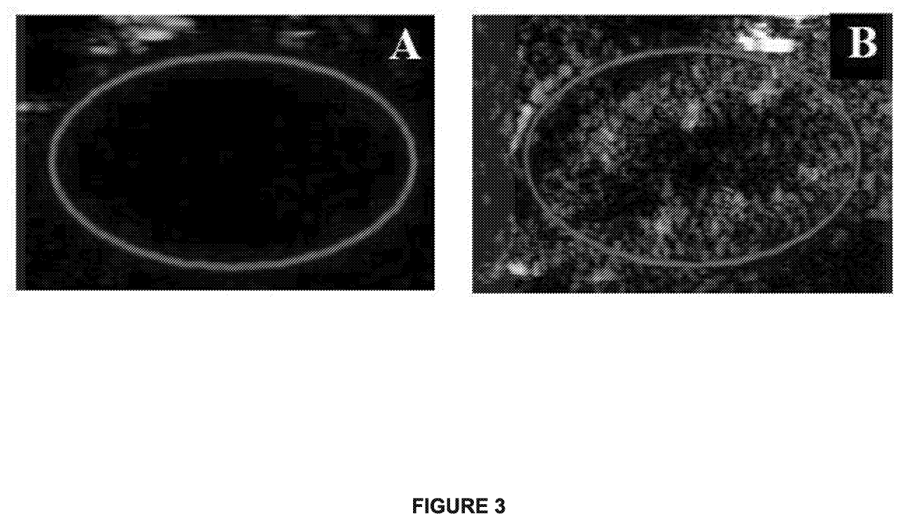

The inventive technology includes the design and production of OMB having novel phospholipid shell compositions. In certain embodiments, OMB may be formed using either 1,2-di stearoyl-sn-gylcero-3-phosphocholine (DSPC) or 1,2-dibehenoyl-sn-glycero-3-phosphocholine (DBPC) for the phospholipid, 1,2-distearoyl-sn-glycero-3-phosphoethanolamine-N-(polyethylene glycol 2000) (DSPE-PEG200) as the emulsifier, and pure oxygen as the gas core. In one embodiment, OMB having variable length acyl-chains were generated, in particular DSPC (C18:0) OMB, and DBPC (C22:0) OMB. As shown in FIG. 3, DBPC microbubbles showed a significant increase in ultrasound contrast persistence in vivo, such that this composition may be suited for intravascular microbubble injection to treat tumor hypoxia in radiation oncology in one embodiment thereof.

The invention provides compositions and methods for the diagnosis and/or treatment of a condition. For example, in one embodiment, a therapeutically effective amount of OMB having a long acyl-chain phospholipid shell may be administered to a subject, such as an animal, and preferably a mammal or human patient. In this embodiment, a subject may receive an initial, repeat or escalating OMB doses.

In another embodiment, a therapeutically effective amount of OMB having a long acyl-chain phospholipid shell may be administered may be delivered to a subject, and more specifically a subject experiencing a disease condition. In this preferred embodiment, a therapeutically effective amount of OMB may be delivered to a cancer cell or tumor, and preferably a hypoxic tumor. The OMB having a long acyl-chain phospholipid shell may be introduced to the subject intravenously, and may be co-administered with another chemotherapeutic treatment, such as an anti-cancer compound, surgery, or radiation.

In another embodiment, a therapeutically effective amount of OMB having a long acyl-chain phospholipid shell may be administered to a subject in need of treatment for a bacterial infection, and preferably the prevention or treatment of one or more bacterial biofilms. The OMB having a long acyl-chain phospholipid shell may be introduced to the subject intravenously, and may be co-administered with an antibiotic.

“Microbubbles” and “bubbles” are used interchangeably herein to refer to a gas core surrounded by a lipid membrane, which can be either a monolayer or a bilayer and wherein the lipid membrane can contain one or more lipids and one or more stabilizing agents. A microbubble may also mean a liposome and/or a micelle.

The term “oxygen microbubble” as used herein means a microbubble having an oxygen gas core.

As used herein “hypoxic tumor” are cancerous cells residing in a hypoxic environment in vivo such as, for example, in a hypoxic tumor zone, or in vitro. Hypoxic tumors may be especially resistant to radiotherapy.

The term “therapeutically effective amount” means an amount of an OMB effective to produce a detectable material, physiological, or diagnostic effect. For example, oxygenation of a hypoxic tumor enhancing its radiosensitivity to radiotherapy, such as X-ray radiotherapy, may be an exemplary physiological effect. Increasing contrast in an ultrasound scan may be an exemplary diagnostic effect. A novel OMB as described herein having increased persistence in vivo may also be an exemplary material effect.

“Acyl” refers to a group having the structure RCO—, where R may be alkyl, or substituted alkyl. The term “alkyl” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 6 carbon atoms. Preferred alkyl groups have 1 to 4 carbon atoms. Alkyl groups may be “substituted alkyls” wherein one or more hydrogen atoms are substituted with a substituent such as halogen, cycloalkyl, alkoxy, amino, hydroxyl, aryl, alkenyl, or carboxyl. For example, a lower alkyl or (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy.

As used herein, the chain length of the “acyl chain” means the number of carbon atoms in the acyl chain. as used herein, “long acyl-chain” refers to an acyl chain having up to 23 carbon atoms.

As used herein, “phospholipid” refers to an organic compound that has two fatty acid moieties attached at the sn-1 and sn-2 positions of glycerol, and contain a head group linked by a phosphate residue at the sn-3 position of the glycerol. Exemplary headgroup moieties include choline, ethanolamine, serine and inositol. Phospholipids include phosphatidyl choline, phosphatidyl serine, phosphatidyl ethanol amine, phosphatidyl inositol and phosphatidic acid. The fatty acid moiety is the portion of the fatty acid molecule that is bound at the sn-1 or sn-2 position, for example by an ester or ether linkage. When the fatty acid moiety is a fatty acyl, the aliphatic chain of the fatty acyl is attached via an ester linkage and when the fatty acid moiety is an aliphatic chain of a fatty acid, the aliphatic chain is attached via an ether linkage.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The term “including” is used herein to mean, and is used interchangeably with, the phrase “including but not limited to.” The term “or” is used herein to mean, and is used interchangeably with, the term “and/or,” unless context clearly indicates otherwise.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1: Oxygen Microbubbles Production and Selection

OMB generated by the present inventors generally comprise a phospholipid shell with a polyethylene glycol (PEG) brush that encapsulates a gas core of pure oxygen as shown in FIG. 1. Longer acyl-chain phospholipids have been shown to stiffen the shell and increase circulation persistence of perfluorocarbon gas microbubbles. Therefore, the present inventors designed novel OMB with longer acyl-chain phospholipid and tested the in vivo stability. The present inventors focused on two acyl-chain phospholipids: shorter chain (C18:0) DSPC and longer chain (C22:0) DBPC. Microbubble volume dose (MVD) was kept constant at 300±10.8 μL/kg throughout the experiments.

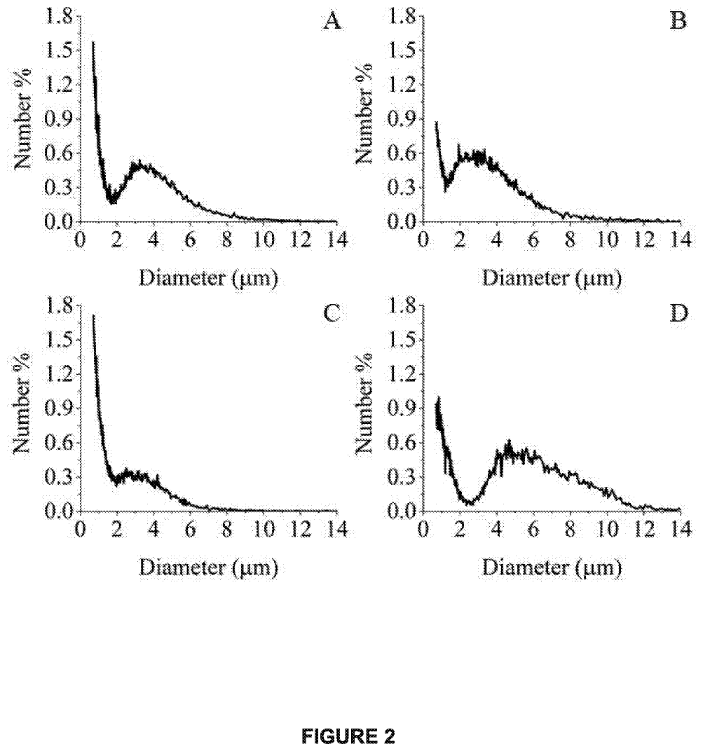

As shown in FIG. 2, size-selection was conducted to obtain OMB between 2-10 μm in diameter. The size distribution was bimodal for both lipid acyl-chain microbubble shell compositions: DSPC (C18:0) and DBPC (C22:0). The first peak had a size ranging approximately from 0.5-2 μm in diameter and the second peak ranged from 2-10 μm in diameter. The initial OMB concentration was measured to be 6.0×10⁹ MB/mL for DSPC and 8.5×10⁹ MB/mL for DBPC. After concentration by centrifugation, the volume fraction was 71% for DSPC and 79% for DBPC. The OMB were diluted using oxygen-saturated PBS to a volume fraction of 50% for in vivo animal injections.

### Example 2: In Vivo Ultrasound Contrast Persistence

In vivo contrast persistence was measured using Vevo 2100 small-animal ultrasound with an 18-MHz transducer placed on the left mouse kidney. All mice were given bolus OMB injections. The MVD was kept constant at 300 μL/kg. OMB have a rapid elimination in vivo, so the bolus injection was not followed with a saline flush. However, the catheter tubing length was taken into account and an additional volume of OMB was injected that filled the catheter tubing length, so the mouse received the OMB full dose.

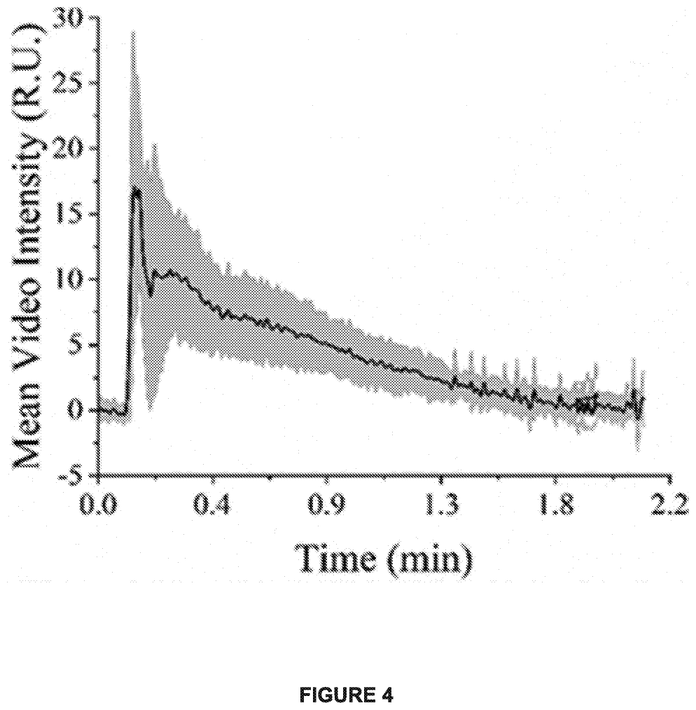

FIG. 3 shows a typical grayscale image of a mouse kidney before and after injection of OMB with the same volume fraction. FIG. 4 demonstrates a post image taken at peak amplitude, a few seconds after injection. Mean video contrast enhancement and persistence in the blood stream was analyzed from the TICs for each OMB. OMB comprised of DSPC did not show a measurable contrast increase above noise. Some sparse contrast was observed, but it could not be accurately quantified due to the low signal-to-noise ratio. However, the attenuation of the signal shows that OMB were present. Injection of DBPC OMB showed a significant increase in contrast and persistence.

The present inventors demonstrate that OMB comprising the long acyl-chain phospholipid, DBPC (C22:0), show an increase contrast enhancement and persistence in vivo. The shorter acyl-chain phospholipid, DSPC (C18:0), showed minimal increase in contrast enhancement and persistence in vivo. This result is consistent with contrast agent studies that compared differing acyl-chain phospholipids for increase contrast persistence and circulation in vitro and in vivo.

### Example 3: Materials and Methods

Materials

Phosphate buffered saline (PBS) solution was prepared by diluting 10× stock solution from Fisher Scientific International, Inc. (Hampton, NH, USA) 9:1 with deionized water (Direct-Q, Millapore, Billerica, MA, USA) and filtered through 0.2 μm diameter nylon filter attached to a vacuum. High purity oxygen was obtained from Airgas (Radnor, PA, USA). The two phospholipids, 1,2-distearoyl-sn-gylcero-3-phosphocholine (DSPC) and 1,2-dibehenoyl-sn-glycero-3-phosphocholine (DBPC) and the emulsifier 1,2-distearoyl-sn-glycero-3-phosphoethanolamine-N-(polyethylene glycol 2000) (DSPE-PEG2000) were purchased from Avanti Polar Lipids (Alabaster, AL, USA).

Microbubble Preparation

Individual lipid solutions were prepared by combining the different acyl-chain phospholipids with DSPE-PEG2000 in a 9:1 molar ratio, respectively, to a final lipid concentration of 12 mg/mL. Lipids were added to PBS and gently stirred using a magnetic stir bar and heated to reach their main phase transition temperature and make a homogenous suspension. The lipid suspension was then sonicated using Branson Digital Sonifier SFX 550 (Danbury, CT, USA) for 10 min at 30% power to disperse lipids into small unilamellar liposomes. The suspension was then cooled to 4° C. before OMB generation.

Oxygen microbubbles were synthesized using an ultrasonic horn reactor enclosed in a water-cooled, continuous-flow chamber (Branson, Danbury, CT, USA). Lipid solutions were flowed through the chamber with room temperature oxygen gas at full sonication power and then collected into a glass collection column with oxygen gas headspace. The final OMB microfoam was collected into 30-mL syringes (BD, Franklin Lakes, NJ, USA) to be further processed.

The collected OMB microfoam was further processed by centrifugation using a bucket-rotor Centrifuge 5804 (Eppendorf, Hauppauge, NY, USA). Microbubble cake was collected by centrifuging the initial MB suspension at 130 relative centrifugal force (RCF) for 1 min. Excess lipid solution from the infranatant was collected and reused to generate more OMB. The final cake was collected into a 30-mL syringe to be size-selected. Differential centrifugation was performed to size-select OMB. The cake was diluted to 30 mL with oxygen saturated PBS and centrifuged at 130 RCF for 1 min to wash out OMB that were smaller than 2-μm diameter. The remaining OMB was transferred to a 12-mL syringe (Covidien Monoject, Mansfield, MA, USA) and washed with PBS once more, with the final product being concentrated OMB.

Characterization of Oxygen Microbubbles

Oxygen microbubbles were characterized by particle size, concentration and gas content. Microbubble size and concentration was measured using an electrozone sensing method (Coulter Multisizer III, Beckman Coulter, Opa Locka, Fl). Size and concentration were measured in triplicate. Gas content was measured using an oxygen headspace sensor (MO-200 Oxygen Sensor, Apogee, Logan, UT). Gas volume fraction was measured by weight and volume of the OMB sample. Microbubble volume dose, i.e., oxygen gas volume injected per weight of animal, was calculated by using OMB concentration, size and total injection volume.

Animal Preparation and In Vivo Injections

All animal experiments were approved by the University of Colorado Denver Institutional Animal Care and Use Committee. Contrast persistence studies were performed in male and female black-6 mice six weeks of age (The Jackson Laboratory). Mice were anesthetized using 2% isoflurane with oxygen carrier gas and placed supine on a heated platform. Heart rate, respiratory rate and temperature were monitored using Vevo 2100

Physiological Monitoring Unit. Mice were kept under anesthesia via nose cone for the duration of the experiment. Hair was removed from the lower left kidney region using Nair Hair Removal Lotion. A modified 27-gauge, one half-inch winged infusion catheter (Terumo, Tokyo, Japan) with tubing removed and replaced with polyethylene tubing (Warner Instruments, Hamden, CT, USA), was placed in the mouse tail vein.

A VisualSonics Vevo 2100 small-animal ultrasound imaging scanner (Toronto, ON, Canada) with an MS250, 18-MHz transducer at 10% power was placed on the shaved kidney region using Medline Aquasonic acoustic coupling gel. Mice were injected with OMB while continuously imaging (n=6-8 per group). Each mouse was imaged once per imaging session and was then removed from anesthesia and placed in a recovery cage on top of a heating pad. Once regaining consciousness, the mouse was returned to its cage.

Data Analysis

The Vevo 2100 small-animal ultrasound provided Digital Imaging and Communications in Medicine (DICOM) files that were used to extract individual video frames and measurements. These files were post-processed using MatLab R2018b (MathWorks, Inc., Natick, MA, USA) DICOM reader and were analyzed for gray scale intensity versus time. For in vivo mouse studies, the kidney was located using β-Mode, and Contrast Mode was used to measure the change in contrast intensity over time. The region of interest (ROI) was chosen as the entire kidney rather than just a portion because signal attenuation and shadowing showed minimal effect. The average video intensity was determined by averaging the intensities over the entire ROI for each frame. The data was baseline adjusted and plotted as mean video intensity versus time. Lower envelope detection in MatLab was used to adjust for respiratory motion of the mouse and produce a smoothed time intensity curve (TIC). Data was fit to an exponential decay model using OriginPro 2019 (OriginLab Corp., Northampton, MA) software. The decay rate was used to determine the half-life of the microbubble. Total integrated signal enhancement (area under the curve, AUC) was measured using the TIC data.

### REFERENCES

The following references are hereby incorporated by reference into the specification:
[1] S. M. Fix et al., “Oxygen microbubbles improve radiotherapy tumor control in a rat fibrosarcoma model—A preliminary study,” PloS One, vol. 13, no. 4, p. e0195667, 2018.
[2] J. R. Eisenbrey et al., “Sensitization of Hypoxic Tumors to Radiation Therapy Using Ultrasound-Sensitive Oxygen Microbubbles,” Int. J. Radiat. Oncol., vol. 101, no. 1, pp. 88-96, May 2018.
[3] J. J. Kwan, M. Kaya, M. A. Borden, and P. A. Dayton, “Theranostic Oxygen Delivery Using Ultrasound and Microbubbles,” Theranostics, vol. 2, no. 12, pp. 1174-1184, 2012.
[4] J. S. Lum, J. D. Dove, T. W. Murray, and M. A. Borden, “Single Microbubble Measurements of Lipid Monolayer Viscoelastic Properties for Small-Amplitude Oscillations,” Langmuir ACS J. Surf. Colloids, vol. 32, no. 37, pp. 9410-9417, 20 2016.
[5] D. H. Kim, M. J. Costello, P. B. Duncan, and D. Needham, “Mechanical Properties and Microstructure of Polycrystalline Phospholipid Monolayer Shells: Novel Solid Microparticles,” Langmuir, vol. 19, no. 20, pp. 8455-8466, Sep. 2003.
[6] S. Garg, A. A. Thomas, and M. A. Borden, “The effect of lipid monolayer in-plane rigidity on in vivo microbubble circulation persistence,” Biomaterials, vol. 34, no. 28, pp. 6862-6870, September 2013.
[7] K.-H. Song, A. C. Fan, J. J. Hinkle, J. Newman, M. A. Borden, and B. K. Harvey, “Microbubble gas volume: A unifying dose parameter in blood-brain barrier opening by focused ultrasound,” Theranostics, vol. 7, no. 1, pp. 144-152, January 2017.
[8] J. A. Feshitan, C. C. Chen, J. J. Kwan, and M. A. Borden, “Microbubble size isolation by differential centrifugation,” J. Colloid Interface Sci., vol. 329, no. 2, pp. 316-324, January 2009.
[9] J. A. Feshitan, N. D. Legband, M. A. Borden, and B. S. Terry, “Systemic oxygen delivery by peritoneal perfusion of oxygen microbubbles,” Biomaterials, vol. 35, no. 9, pp. 2600-2606, March 2014.
[10] V. Baranau and U. Tallarek, “Random-close packing limits for monodisperse and polydisperse hard spheres,” Soft Matter, vol. 10, no. 21, pp. 3826-3841, May 2014.
[11] R. S. Farr and R. D. Groot, “Close packing density of polydisperse hard spheres,” J. Chem. Phys., vol. 131, no. 24, p. 244104, December 2009.
[12] S. Sirsi, J. Feshitan, J. Kwan, S. Homma, and M. Borden, “Effect of Microbubble Size on Fundamental Mode High Frequency Ultrasound Imaging in Mice,” Ultrasound Med. Biol., vol. 36, no. 6, pp. 935-948, June 2010.

1. A novel microbubble composition comprising a lipid outer shell encasing an gas core wherein said lipid outer shell comprises a plurality of long acyl-chain phospholipids

2. The composition of claim 1 and further comprising a polyethylene glycol (PEG) brush structure.

3. The composition of claim 1 wherein said plurality of long acyl-chain phospholipids comprises a plurality of long acyl-chain phospholipids having less than 24 carbons in the acyl-chain.

4. The composition of claim 1 wherein said plurality of long acyl-chain phospholipids comprises a plurality of long acyl-chain phospholipids having at least 22 carbons in the acyl-chain.

5. The composition of claim 1 wherein said long acyl-chain phospholipid comprises 1,2-dibehenoyl-sn-glycero-3-phosphocholine (DBPC).

6. The composition of claim 1 wherein said long acyl-chain phospholipid comprises 1,2-distearoyl-sn-gylcero-3-phosphocholine (DSPC)

7. The composition of claim 1 wherein said microbubble is formed using an emulsifier.

8. The composition of claim 7 wherein said emulsifier comprises 1,2-distearoyl-sn-glycero-3-phosphoethanolamine-N-(polyethylene glycol 2000) (DSPE-PEG2000).

9. The composition of claim 1 wherein said novel microbubble comprises a microbubble having increased ultrasound contrast enhancement and persistence in vivo and in vitro, and enhanced stability.

10. The composition of claim 1 wherein said gas core comprises an oxygen gas core.

11. The composition of claim 10 wherein said oxygen gas core comprises a pure oxygen gas core.

12. The composition of claim 11 wherein said novel microbubble is between 0.1-10 μm in diameter.

13. The composition of claim 11 wherein said novel microbubble is larger than 10 μm in diameter.

14. A method of treating a hypoxic tumor, comprising administering a therapeutically effective amount the microbubble composition of claim 1 to a patient in need thereof.

15. (canceled)

16. A method of treating a bacterial infection, comprising administering a therapeutically effective amount the microbubble composition of claim 1 to a subject in need thereof.

17. The method of claim 16 wherein said bacterial infection comprises a bacterial biofilm.

18. The method of claim 14 wherein said step of administering comprises administering a therapeutically effective amount of said microbubble composition intravenously to a subject in need thereof.

19. The method of claim 14 further comprising the step of applying ultrasound radiation to said composition in vivo.

20. The method of claim 19 wherein said subject has a hypoxic tumor that is oxygenated by said microbubble composition, and further applying therapeutic radiation to said oxygenated hypoxic tumor.

21. The method of claim 20 wherein said therapeutic radiation comprises x-ray radiation.
